(19) United States
(12) Patent Application Publication
(10) Pub. No.: US 2008/0161425 A1
(43) Pub. Date: Jul. 3, 2008

(54) Regulation of Tissue Factor Activity by Protein S and Tissue Factor Pathway Inhibitor

(75) Inventors: Tilman M. Hackeng, Maastricht (NL); Kristin M. Sere, Oirsbeek (NL); Guido Tans, Maastricht (NL); Jan Rosing, Maastricht (NL)

Correspondence Address: KALOW & SPRINGUT LLP, 488 MADISON AVENUE, 19TH FLOOR, NEW YORK, NY, 10022, US

(73) Assignee: Universiteit Van Maastricht, Maastricht (NL)

(21) Appl. No.: 11/997,289

(22) PCT Filed: Jul. 28, 2006
(86) PCT No.: PCT/EP2006/007597

§ 371 Date: Feb. 29, 2008

(30) Foreign Application Priority Data
Jul. 29, 2005 (EP) 05076763.1

(51) Int. Cl. A61K 47/00; C12Q 1/56; G01N 33/00; G01N 33/53; G01N 33/566

(52) U.S. Cl. 514/789; 435/13; 436/86; 435/7.1; 436/501

(57) ABSTRACT

The present invention relates to methods for the identification of compounds that increase or decrease the inhibitory effect of TFPI on tissue factor activity and/or Factor Xa activity and/or thrombin formation. The invention also relates to methods for the identification of compounds that increase or decrease the co-factor activity of Protein S in TFPI-mediated inhibition of tissue factor and/or Factor Xa activity. This invention also relates to a pharmaceutical composition comprising the compounds identifiable by such methods. The invention also relates to methods for the regulation of tissue factor activity by influencing the interaction between Protein S and Tissue Factor Pathway Inhibitor.

### FIELD OF THE INVENTION

The present invention is based on the finding that Protein S is involved in the regulation of tissue factor (TF) activity, wherein Protein S acts as a co-factor to Tissue Factor Pathway Inhibitor (TFPI). Hence, the invention is in the field of biochemistry and medicine, and relates in particular to methods of treatment and/or prophylaxis of diseases or disorders associated with tissue factor activity, especially in blood. More in particular, the present invention relates to methods for the identification of compounds that increase or decrease the inhibitory effect of TFPI on tissue factor activity and/or Factor Xa activity and/or thrombin formation. The invention also relates to methods for the identification of compounds that increase or decrease the co-factor activity of Protein S in TFPI-mediated inhibition of tissue factor and/or Factor Xa activity. The invention also relates to a pharmaceutical composition comprising the compounds identifiable by such methods. The invention also relates to methods for the regulation of tissue factor activity by influencing the interaction between Protein S and Tissue Factor Pathway Inhibitor.

### BACKGROUND OF THE INVENTION

Tissue factor (TF) is a multifunctional protein that is not only involved in haemostasis, thrombosis (1) and atherosclerosis (2), but also participates in cell signaling activities (3, 4) that play an important role in inflammation (5) and angiogenesis (6, 7). Historically, TF was identified as the protein component in tissue extracts that is responsible for the initiation of blood coagulation. Upon exposure to blood, TF binds the circulating coagulation factor VIIa (FVIIa). The resulting phospholipid-bound TF/FVIIa complex converts the zymogen factor X into the active serine protease, factor Xa (FXa). Together with its cofactor factor Va (FVa), FXa subsequently incorporates into the prothrombinase complex and activates prothrombin to thrombin.

Coagulation is finely tuned and during thrombin formation several anti-coagulant reactions are initiated to prevent systemic activation of coagulation. Impaired activity of the anticoagulant systems results in a hypercoagulable state and increases the risk of venous thrombosis (8). The present invention relates to two natural anticoagulant proteins, tissue factor pathway inhibitor (TFPI) and protein S, deficiencies of which are associated with venous thrombosis (9, 10).

TFPI is a Kunitz-type inhibitor that inhibits TF/FVIIa initiated coagulation (11) via a two step feed-back mechanism through formation of a bimolecular FXa/TFPI complex that subsequently interacts with TF/FVIIa, yielding an inactive quaternary complex and resulting in termination of TF/FVIIa-catalyzed FX activation (12).

Protein S is an essential component of the protein C pathway which down-regulates thrombin formation (13). Activated protein C (APC) is a serine protease that inhibits thrombin generation via inactivation of the coagulation factors Va and VIlla. Protein S is a cofactor in these reactions which enhances the anticoagulant activity of APC up to twenty-fold (14, 15).

It has been described that Protein S can also down-regulate thrombin generation in the absence of APC via a mechanism that is hitherto not understood (19). Since protein S directly inhibits prothrombin activation in model systems, it is generally thought that protein S exerts its anticoagulant activity in the absence of APC via direct interactions with FXa, FVa and phospholipids (16-18). Currently, there is no study that reveals the mechanism underlying the effect of protein S on the coagulation system and on the activity of tissue factor activity in the absence of APC in plasma.

It would be desirable to be able to interfere with the APC independent effect of protein S on the coagulation pathway in order to provide new treatments for haemostatic disorders thrombosis and atherosclerosis as well as bleeding disorders related to an impaired coagulation system, such as hemophilia.

### SUMMARY OF THE INVENTION

The present invention is, to some extent, based on a hitherto unrecognized interplay between TFPI and protein S in the inhibition of TF activity and Factor Xa activity. The clear insight in the newly discovered mechanism that involves protein S as a co-factor for TFPI in down regulating TF-activity and Factor Xa activity in plasma now allows the identification and development of specific pharmaceutical compounds that interfere with or improve this Protein S co-factor activity.

In one aspect, the invention relates to a method for the identification of a compound that improves or decreases the inhibitory effect of TFPI on tissue factor activity and/or Factor Xa activity and/or thrombin formation.

In another aspect, the invention relates to a method for the regulation of tissue factor activity and/or Factor Xa activity by influencing the interaction between Protein S and TFPI.

In yet another aspect, the invention relates to the use of a TFPI and/or Protein S antagonist identifiable or identified by the methods described above for the preparation of a medicament for increasing the coagulation potential of blood.

In yet another aspect, the invention relates to the use of a TFPI and/or Protein S agonist identifiable or identified by the methods described above for the preparation of a medicament for decreasing the coagulation potential of blood.

The invention also relates to a method for the preparation of a medicament for the treatment of a thrombotic disorders comprising the steps of:
a. Identifying a compound that improves the inhibitory effect of TFPI on tissue factor activity and/or Factor Xa activity and/or thrombin formation by a method described above and
b. mixing the compound identified in step a) with a pharmaceutically acceptable carrier.

The invention also relates to a method for the preparation of a medicament for the treatment of bleeding disorders comprising the steps of:
a. Identifying a compound that decreases the inhibitory effect of TFPI on tissue factor activity and/or Factor Xa activity and/or thrombin formation by a method described above and
b. mixing the compound identified in step a) with a pharmaceutically acceptable carrier.

In yet another aspect, the invention relates to the use of a compound capable of increasing or decreasing the co-factor activity of Protein S on TFPI for the preparation of a medicament for the treatment of diseases associated with tissue factor activity in blood.

Such diseases may be disorders wherein the coagulation is impaired so that the tendency to clot is too strong (e.g. thrombosis) or too weak (e.g. hemophilia). In one aspect of the present invention the medical condition associated with tissue factor activity in blood is selected from the group consisting of thrombosis and haemostasis and related disorders.

In another aspect of the present invention the medical condition associated with tissue factor activity in blood is selected from the group consisting of cancer, inflammation and cardiovascular disorders.

In yet another aspect of the present invention the medical condition associated with tissue factor activity in blood is selected from the group consisting of bleeding disorders such as hemophilia and related disorders.

These and other aspects of the present invention will now described in more detail below.

### BRIEF DESCRIPTION OF THE DRAWINGS

FIG. 1: Thrombin generation in plasma. Panel A: Thrombin generation was initiated in plasma (in the presence of APC-inhibiting antibodies) with 1.4 pM TF, 10 μM phospholipid vesicles and 16 mM CaCl₂ (final concentrations) and followed continuously with the fluorogenic substrate I-1140 (Z-Gly-Gly-Arg-AMC.HCl). () normal plasma with protein S; (◯) normal plasma without protein S; (▴) TFPI-depleted plasma with protein S; (Δ) TFPI-depleted plasma without protein S. A typical experiment is shown. Panel B: ETP values of TFPI-depleted plasma reconstituted with varying amounts of full length TFPI (,◯) or TFPI₁₋₁₆₁ (▴,Δ) in the presence (closed symbols) or absence of protein S (open symbols). The averages of two independent experiments are shown.

FIG. 2: Inhibition of TF/FVIIa-catalyzed FX-activation by full length TFPI and protein S. Activation of 160 nM FX by 1 pM TF/FVIIa was followed in reaction mixtures which contained 15 μM phospholipids, 3 mM Ca²⁺ and (♦) no TFPI and no protein S, (▪) 100 nM protein S, () 1 nM TFPI, (▴) 1 nM TFPI and 100 nM protein S. Averages of three independent measurements ± standard deviation are shown. Note that the curves of (♦) and (▪) overlap at least partially.

FIG. 3: Influence of protein S on FXa inhibition by full length TFPI.

Panel A: Conversion of 0.5 mM S2222 by 0.2 nM FXa was monitored in reaction mixtures containing 10 μM phospholipids, 3 mM CaCl₂ and 0 (dashed line) or 160 nM (dotted line) protein S. Without TFPI, S2222 conversion by FXa was linear in time (with- or without protein S present, solid line). At the time indicated 1.54 nM TFPI was added. The absorbance data were fitted to equation d. Panel B: First derivatives of the fitted curves representing the change in free FXa with time. A typical experiment is shown in panel A and B.

FIG. 4: Effect of protein S on vₒ and vₛ calculated from time courses of FXa inhibition by TFPI. Progress curves of S2222 conversion by FXa were measured at varying concentrations TFPI in the absence and presence of protein S. Fitting of the progress curves to equation (d) yielded values for vₒ (A) and vₛ (B) as function of the TFPI concentration. Final concentrations were 0.2 nM FXa, 10 μM phospholipid vesicles (20/60/20 DOPS/DOPC/DOPE), 3 mM CaCl₂, 0.5 mM S2222, 0-3.9 nM TFPI and 0 nM (◯) or 100 nM () protein S. The average of two independent experiments is shown.

### DETAILED DESCRIPTION OF THE INVENTION

The present invention was triggered by the finding that protein S, as a co-factor, is involved in the regulation of tissue factor activity in blood. More specifically, it was discovered by the inventors that protein S and TFPI act in concert in the inhibition of tissue factor activity. It is therefore concluded that Protein S deficiency not only increases the risk of thrombosis by impairing the protein C system but also by reducing the ability of TFPI to down-regulate the extrinsic coagulation pathway.

Since tissue factor is also involved in cell signaling in angiogenesis and inflammation, these findings now allow for the identification and development of compounds useful for the treatment and prophylaxis of angiogenesis and inflammation.

The results presented here provide new insight in the mechanism via which TF-activity is regulated in blood. Protein S inhibits TF-activity by enhancing the interaction between TFPI and FXa thereby accelerating the feed-back inhibition of the extrinsic TF/FVIIa pathway by TFPI. This observation not only underscores the important role of protein S in the down-regulation of coagulation, but also provides a mechanistic basis of the APC-independent anticoagulant activity of protein S in blood (19).

These new insights now allow for the identification and isolation of compounds that specifically interfere with this mechanism. In one aspect, compounds may now be developed that mimic or improve the co-factor activity of Protein S on TFPI. Such compounds may then be advantageously used in the treatment of thrombosis and haemostasis and related disorders where it is required to decrease the clotting potential of blood.

The new insights also allow for the identification and isolation of compounds that decrease or abolish the co-factor activity of Protein S on TFPI. Such compounds may then be advantageously used in the treatment of bleeding disorders such as hemophilia.

What follows is a detailed description of the mechanism wherein protein S acts as a co-factor for TFPI.

Ever since the first report on the inhibition of prothrombin activation by protein S in the absence of APC (23), this inhibition was explained by direct interactions of protein S with the components of the prothrombinase complex FVa, FXa and phospholipids (16-18). Since then, in purified protein S preparations in vitro generated protein S multimers have been identified that bind with a high affinity to phospholipids (K_{d}<1 nM) and account for the effective inhibition of prothrombin activation by protein S in model systems (24). However, protein S multimers are absent in plasma (24), and it was proposed that the APC-independent inhibition of thrombin generation in plasma by protein S is not due to competition between protein S and other coagulation factors for binding to procoagulant membrane surfaces (19).

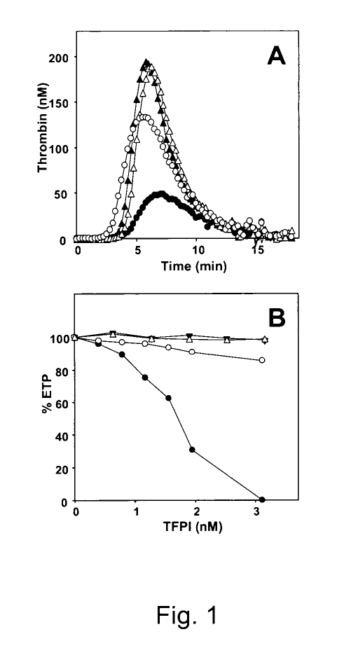

The present inventors demonstrate that the APC-independent inhibition of thrombin generation by protein S in plasma is also not explained by inhibition of prothrombin activation via direct interactions of protein S with FXa and FVa. The observations that protein S does not inhibit thrombin generation in TFPI deficient plasma (FIG. 1A) and that TFPI is a very poor inhibitor of thrombin generation in the absence of protein S (FIG. 1B) led to the hypothesis that protein S acts as a cofactor of TFPI in the inhibition of TF/FVIIa-catalyzed FX activation. The partial activity (60%) of the protein S—C4BP complex is not yet understood, and can originate from a change in phospholipid-binding affinity of the complex, or from sterical hindrance by C4BP when protein S is in complex with C4BP.

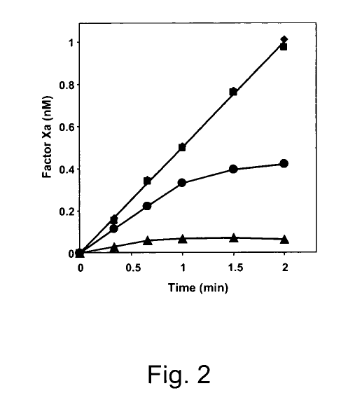

Experiments in a model system confirmed that protein S enhances the inhibition of TF/FVIIa-catalyzed FX activation by TFPI (FIG. 2). The inhibition of FX activation by TFPI involves the formation of a FXa/TFPI complex which slowly isomerizes into a tight FXa/TFPI* complex (21) that subsequently forms an inactive quaternary complex with TF/FVIIa (20). Detailed kinetic analysis showed that protein S enhances the formation of the FXa/TFPI complex and has a minor effect on the subsequent isomerization step.

The stimulatory effect of protein S on FXa inhibition by TFPI is due to a 10-fold reduction of the Kᵢ of the FXa/TFPI complex, which decreased from 4.4 nM in the absence to 0.5 nM in the presence of protein S, No test was performed whether protein S may also have an effect on the formation of the quaternary complex. However, since the formation of this complex is very fast and diffusion limited (20), it is unlikely that this step is affected by protein S.

Without wanting to be bound by theory, the inventors provide the following observations on the mechanism by which protein S enhances the formation of the FXa/TFPI complex. The fact that protein S only acts as a cofactor of TFPI in the presence of phospholipids suggests that co-localization and/or juxtaposition of protein S, TFPI and FXa on the phospholipid surface is a prerequisite for the fast protein S-mediated inhibition of FXa by TFPI. Protein S did not stimulate the inhibition of FXa by truncated TFPI (TFPI₁₋₁₆₁), a form of TFPI that lacks the Kunitz-3 domain and the C-terminus. It was reported that Kunitz-3 and the C-terminus of TFPI are involved in the binding of full length TFPI (25) to cell surfaces and that the C-terminus interacts with anionic phospholipids (26) and with the GIa-domain of FXa (27). Hence, the loss of these interaction sites of TFPI₁₋₁₆₁ may explain why protein S does not stimulate the inhibition of FXa by TFPI₁₋₁₆₁ (Table 2) and why truncated TFPI lacks inhibitory activity in plasma (FIG. 1B) (27-29).

Both TFPI and protein S play an important role in the in vivo down-regulation of coagulation. This is illustrated by the observations that mice with a mutant form of TFPI that did not bind FVIIa died intra-uterine or during the neonatal period due to consumptive coagulopathy (30) and that homozygous protein S deficiency, which is also lethal if left untreated, presents with a similar phenotype of consumptive coagulopathy (31). Furthermore, population-based studies indicated that low levels of protein S (9) and TFPI (10) are associated with an increased risk of venous thrombosis. In view of the pivotal role of TFPI and protein S in the regulation of coagulation, it is not surprising that our observations have important physiological implications. Considering its effect on the Kᵢ for the inhibition of FXa by TFPI, which decreases from 4.4 nM in the absence to 0.5 nM in the presence of protein S, protein S brings the TFPI concentration necessary for efficient down-regulation of extrinsic FX activation well within the range of the free TFPI concentration in plasma (0.25-0.5 nM) (32). Using the equations for a simple binding equilibrium, it can be calculated that, during the initiation of coagulation (where [FXa]<[TFPI]), protein S reduces the free FXa concentration from 90% to 45%, and thus increases the concentration of FXa/TFPI complex approximately 5-fold. This means that protein S enhances the down-regulation of thrombin formation by 1) reducing the amount of FXa that can participate in prothrombin and FVII activation and 2) by increasing the amount of FXa/TFPI complex available for inhibition of the TF/FVIIa complex.

The extent of inhibition of the extrinsic coagulation pathway by TFPI depends on the TF concentration and the amount of FXa that escapes regulation by TFPI linearly increases with the TF concentration (20). This means that at increasing amounts of TF, TFPI will ultimately fail to keep the FXa concentration below the threshold required for thrombin formation (33), which explains why protein S hardly inhibits thrombin generation at high TF concentrations (19). Thus, protein S and TFPI likely play a prominent role in suppressing the procoagulant activities at low tissue factor concentrations e.g. of the small amounts of TF (˜3 pM) circulating in plasma (34). This means that protein S deficiency affects the two cofactor activities of protein S: the TFPI-cofactor activity at low TF concentrations and the APC-cofactor activity at high TF-concentrations (19). On the basis of our observations we propose that the increased risk of venous thrombosis associated with protein S deficiency may in part be explained by an impaired down-regulation of the extrinsic coagulation pathway by TFPI at low protein S concentrations.

In addition to its role in haemostasis, TF is also involved in inflammation (5), angiogenesis (6, 7) and tumor metastasis (35), processes that are likely modulated through TF/FVIIa- and TF/FVIIa-FXa-dependent PAR signaling (3, 4, 36-38). Recently, a selective role for TFPI was proposed in the inhibition of TF signaling through PAR1 and PAR2, in which PAR1 signaling appeared less sensitive to inhibition TFPI than was PAR2(39). Whether protein S also affects these functions of TF, especially the inhibition of TF-mediated PAR1 signaling by TFPI, remains to be elucidated.

By virtue of the new insights in the mechanism of anti-coagulation activity of Protein S as described above, the following inventions now become available for the skilled person.

The invention relates to a method for the identification of a compound that improves or decreases the inhibitory effect of TFPI on tissue factor activity and/or Factor Xa activity and/or thrombin formation. Such a method is now enabled through the discovery of the mechanism wherein protein S acts as a co-factor for TFPI in down regulating TF-activity and Factor Xa activity in blood.

Specific substances may now be designed and tested for their ability to interfere with this newly discovered mechanism. Alternatively, substances known to interfere with the activity of Protein S, TFPI, Factor Xa and Tissue Factor may now be tested for their potential to interfere with the newly discovered mechanism.

Compounds that increase or improve the inhibitory effect of TFPI are herein further also referred to as agonists of TFPI, compounds that decrease the inhibitory effect of TFPI are herein further also referred to as antagonists of TFPI. It is to be understood that neither agonists nor antagonists have to exert their action directly on TFPI, their action may also be directed towards an inhibitor, stimulator of co-factor of TFPI.

There are a vast number of assays available for the identification of a compound that improves or decreases the inhibitory effect of TFPI on tissue factor activity and/or Factor Xa activity and/or thrombin formation. First, activity assays for tissue factor activity and/or Factor Xa activity are widely used in the field and are even commercially available. Secondly, assays for the determination of thrombin formation are also known in the field and are also commercially available.

Since it now has been discovered that Protein S acts as a co-factor for TFPI-mediated inhibition of tissue factor activity and for TFPI-mediated inhibition of factor Xa activity such assays may be performed in the presence of protein S as exemplified herein, and inhibitory or stimulating compounds may thus be identified.

In order to perform such a method, the skilled person would apply for instance a test for the TFPI-mediated inhibition of TF/FVIIa catalyzed activation of FX as described in Examples 3 and 7 and depicted in FIG. 2. He then would test whether the addition of certain compounds would improve or decrease the inhibitory effect of TFPI on Factor Xa and/or T/FVIIa.

The skilled person may also choose to use alternative methods. As exemplified in Examples 4 and 8, he may use a Factor Xa activity assay to determine the inhibition of Factor Xa by TFPI in the presence or absence of Protein S. He then would test whether the addition of certain compounds would improve or decrease the inhibitory effect of TFPI on Factor Xa.

In yet another alternative, the skilled person may employ a thrombin generation assay as exemplified in examples 2 and 6 and depicted in FIG. 1. He then would test whether the addition of certain compounds in the presence or absence of Protein S would improve or decrease the inhibitory effect of TFPI on thrombin formation.

Alternatively, direct binding assays such as label-free surface plasmon resonance (SPR) based technology for studying biomolecular interactions (BiaCore) may also be successfully employed to study the molecular interactions between Protein S, TFPI, Factor Xa, Tissue Factor and Factor VIIa.

The invention thus relates to a method as described above wherein the inhibitory effect of TFPI is measured in an assay for tissue factor activity and/or Factor Xa activity, and/or an assay for thrombin formation, in the presence of protein S.

The skilled person will appreciate that the same assay may then be repeated in the absence of Protein S in order to assure that the measured effect is not due to an artifact or to a mechanism unrelated to Protein S.

In more detail, a particularly advantageous and simple way to identify agonists or antagonists is to add a number of potential agonists or antagonists in the methods as described above and determine whether they influence or interfere with the inhibitory effect of TFPI.

The invention therefore relates to a method as described above wherein the inhibitory effect of TFPI is measured in the presence and absence of a potential TFPI agonist or antagonist and the level of TFPI inhibition in the presence of the potential agonist or antagonist is compared with the level of TFPI inhibition in the absence of the potential TFPI agonist or antagonist.

Potential agonists or antagonists may be newly designed or be selected from a group of already known substances known to interfere with the components that have now been identified to play a role in the newly discovered mechanism. Now that the molecular mechanism of TFPI-mediated inhibition of tissue factor activity and Factor Xa activity is known, antibodies against Protein S, TFPI, Tissue Factor, Factor Xa and Factor VIIa are likely candidates for agonists or antagonists of that mechanism.

The invention therefore relates to a method as described above wherein the potential TFPI agonist or antagonist is selected from the group consisting of antibodies against Protein S, antibodies against TFPI, antibodies against tissue factor, antibodies against Factor VIIa, antibodies against Factor Xa, fragments or analogues of TFPI, fragments or analogues of Protein S, fragments or analogues of Factor Xa, fragments or analogues of Factor VIIa and fragments or analogues of Tissue Factor.

Such antibodies may advantageously be monoclonal, polyclonal, bi-specific or single chain. A particularly useful antibody would be a bi-specific antibody with affinity for Protein S as well as TFPI.

Because of their ease-of-use, the above methods may advantageously be performed using a chromogenic or fluorogenic substrate.

The methods described above may also be advantageously used to identify a compound that is capable of increasing or decreasing the co-factor activity of Protein S in TFPI-mediated inhibition of tissue factor activity and/or Factor Xa activity and/or thrombin formation.

In a particularly advantageous embodiment, the method according to the invention can be used to identify compounds that specifically interfere with the co-factor activity of Protein S in TFPI-mediated inhibition of tissue factor activity and/or Factor Xa activity and/or thrombin formation. This means that such compounds will not interfere with the co-factor activity of Protein S in the APC pathway. To that end, the compounds identified in the methods as described above may be tested for activity in an APC activity assay, for instance an assay wherein the Protein S stimulated inactivation of Factor Va is measured. Advantageously, this APC activity is measured using recombinant Factor Va derivatives such as Factor Va Leiden.

The invention therefore also relates to a method as described above wherein the compound that specifically improves or decreases the inhibitory effect of TFPI on tissue factor activity and/or Factor Xa activity and/or thrombin formation does not interfere with the co-factor activity of Protein S on the APC system.

Compounds that may be tested for their potential to improve or decrease the co-factor activity of Protein S on TFPI are fragments or derivatives of Protein S and/or TFPI and/or Factor Xa. Such derivatives may be obtained by random mutagenesis of Protein S and/or TFPI and/or Factor Xa or other suitable methods known in the art.

Also antibodies against TFPI may be able to enhance the inhibitory effect of TFPI on tissue factor activity. A skilled person would know how to generate antibodies against TFPI and would be able to test them for activity in one of the assays as described above.

Now that the mechanism of TFPI-mediated inhibition of tissue factor and Factor Xa has been revealed, this may advantageously be used to interfere with tissue factor activity and/or Factor Xa activity. The invention therefore also relates to a method for the regulation of tissue factor activity and/or Factor Xa activity by influencing the interaction between Protein S and TFPI. For that purpose, the compounds as identified in the methods as described above may advantageously be used.

Consequently, the invention relates to such a method wherein a compound such as an agonist or antagonist of TFPI is used that is identifiable or identified by a method as described above.

A TFPI and/or Protein S antagonist identifiable or identified by a method as described above may be advantageously used for the preparation of a medicament for increasing the coagulation potential of blood. This is particularly useful for the treatment of patients with a low level of tissue factor.

Levels of tissue factor cannot be expressed in absolute concentrations since it acts as a membrane-bound protein that is locally exposed, e.g. at sites of injury. A low level of tissue factor is defined as a level insufficient to cause effective coagulation. The skilled person will appreciate that such levels may even differ between patients. For instance, the level of tissue factor that is sufficient to cause thrombus formation in normal subjects, is insufficient to cause such thrombus formation in hemophiliacs.

A TFPI and/or Protein S antagonist identifiable or identified by a method as described above may also be particularly useful for the treatment of patients with bleeding disorders such as hemophilia.

In the inverse, the invention relates to the use of a TFPI and/or Protein S agonist identifiable or identified by a method as described above for the preparation of a medicament for decreasing the coagulation potential of blood.

This is particularly useful for the treatment of patients with thrombotic disorders such as deep venous thrombosis.

Defined in another way, the invention relates to a method for the preparation of a medicament for the treatment of thrombotic disorders comprising the steps of:

a) Identifying a compound that improves the inhibitory effect of TFPI on tissue factor activity and/or Factor Xa activity and/or thrombin formation by a method described above and
b) mixing the compound identified in step a) with a pharmaceutically acceptable carrier.

In the inverse, the invention relates to a method for the preparation of a medicament for the treatment of bleeding disorders comprising the steps of:

a) Identifying a compound that decreases the inhibitory effect of TFPI on tissue factor activity and/or Factor Xa activity and/or thrombin formation by a method as described above and
b) mixing the compound identified in step a) with a pharmaceutically acceptable carrier.

Described in yet another way, the invention relates to the use of a compound capable of increasing or decreasing the co-factor activity of Protein S in TFPI-mediated inhibition of tissue factor for the preparation of a medicament for the treatment of diseases associated with tissue factor activity in blood.

Such diseases associated with tissue factor activity in blood may be selected from the group consisting of thrombosis and haemostasis and related disorders. In another aspect of the present invention the diseases associated with tissue factor activity in blood may be selected from the group consisting of cancer, inflammation and cardiovascular disorders. In yet another aspect of the present invention the diseases associated with tissue factor activity in blood may be selected from the group consisting of bleeding disorders such as hemophilia and related disorders.

In certain medical conditions associated with tissue factor activity in blood, such as bleeding disorders it may be desirable to improve the clotting potential of blood, such as in particular hemophilia. For the treatment of such disorders, it may be desirable to decrease the activity of protein S and TFPI in order to increase the activity of tissue factor and/or Factor Xa in blood and/or plasma. Therefore, the invention also provides a pharmaceutical composition for the prophylaxis or treatment of a medical condition associated with tissue factor activity in blood, comprising a protein S and/or TFPI antagonist. Such an antagonist preferably is an antibody or fragment thereof, that specifically binds to protein S or TFPI and thereby specifically interferes with the co-factor activity of Protein S in reactions that involve TFPI. The antibody may advantageously be a polyclonal antibody or a monoclonal antibody.

A diverse number of compounds may be suitable to obtain such an effect. First, a fragment of Protein S may act as a co-factor for TFPI. Suitable fragments of Protein S may be generated by proteolytic treatment of Protein S but also by peptide synthesis. Several overlapping peptides may be generated and tested for activity in one of the above described assays. Second, a number of antibodies is already available and may also be generated that can be tested for the desired activity. Third, fragments of TFPI may interact with the co-factor activity of Protein S and thereby effectively abolish this co-factor activity. Advantageously, compounds may be selected that interfere specifically with the co-factor activity of Protein S on TFPI, which means that such compounds would preferably not interfere with the anti-coagulant activity of Protein S on the APC system.

A substance identified by a method as described above, such as for instance a Protein S antagonist, may therefore be advantageously used for the preparation of a medicament for increasing the coagulation potential of blood. A protein S agonist may on the other hand be used for the preparation of a medicament for decreasing the coagulation potential of blood

### EXAMPLES

### Example 1

### Materials

Hepes-buffer was obtained from Sigma (St Louis, Mass.); Bovine serum albumin (BSA) from ICN (Aurora, Ohio); Fluorogenic substrate I-1140 was from Bachem (Switzerland); Recombinant tissue factor (thromboplastin) was from Dade Innovin (Dade Behring, Marburg, Germany); 1,2-Dioleoyl-sn-glycero-3-phosphocholine (DOPC), 1,2-Dioleoyl-sn-glycero-3-phosphoserine (DOPS) and 1,2-Dioleoyl-sn-glycero-3-phospho-ethanolamine (DOPE) were obtained from Avanti Polar Lipids (Alabaster, Ala.). Phospholipids vesicles (20% DOPS, 20% DOPE, 60% DOPC) were prepared as described previously (19).

Polyclonal anti protein S and anti protein C antibodies were obtained from DAKO (Glostrup, Sweden). Human factor Xa was obtained from Enzyme Research Laboratories (South Bend, Ind.). TFPI was kindly provided by Dr Lindhout from our institute (40). Full length TFPI was produced in Escherichia coli, the truncated variant of TFPI (amino acid residues 1-161) was expressed in Sacharomyces cerevisiae. Purification and analysis of both forms of TFPI has been described previously (41, 42). The TFPI concentration was determined as described (43). Recombinant FVIIa (NovoSeven) was obtained from Novo Nordisk. TFPI-depleted plasma was a kind gift of Dr van Oerle from our institute. The plasma was depleted from TFPI as described by Van 't Veer et al (44): normal pooled plasma was applied to an antibody column containing rabbit polyclonal antibodies directed against the N-terminal region of TFPI. The remaining TFPI activity (<1%) was determined with an in-house assay which is based on the chromogenic assay described by Sandset et al (45).

### Example 2

### Measurement of Thrombin Generation

Thrombin generation was initiated in normal pooled plasma with 1.4 pM TF, 10 μM phospholipid vesicles (20/60/20 DOPS/DOPC/DOPE) and 16 mM CaCl₂ (final concentrations) and continuously followed with the fluorogenic substrate I-1140 (Z-Gly-Gly-Arg-AMC.HCl) as previously described (19, 46). Interference by APC-activity was excluded in all experiments by addition of inhibitory anti-(activated) protein C antibodies (1.23 μM IgG) sufficient to completely block both activation of endogenous protein C and the effect of 5 nM activated protein C added to plasma. Protein S was inhibited in plasma by addition of saturating amounts of polyclonal antiserum against protein S (2.73 μM IgG) and preincubation of plasma during 15 min at 37° C. prior to the initiation of thrombin generation as described in reference (19). When indicated, C4BP was added to plasma to a final concentration of approx 575 nM (approx 200 nM endogenous C4BP and 375 nM exogenous C4BP) and incubated for 30 min prior to the addition of anti protein C antibodies with or without antibodies against protein S. The endogenous thrombin potential (ETP=area under the curve) was calculated from thrombin generation curves by means of the calibrated automated thrombogram (CAT) computer software provided by Synapse BV (Maastricht, The Netherlands)(46).

### Example 3

### Inhibition of TF/FVIIa-catalyzed activation of FX by TFPI and protein S

1 pM TF was incubated with 500 pM recombinant FVIIa (NovoSeven) in the presence of 15 μM phospholipids (20/60/20 DOPS/DOPC/DOPE) at 37° C. in Hepes-buffered saline (HBS: 25 mM Hepes, 175 mM NaCl, pH 7.7) containing 3 mM CaCl₂ and 0.5 mg/ml BSA. FXa generation was started by addition of 160 nM human FX either in the absence or presence of 1 nM TFPI and/or 100 nM protein S (final concentrations). After different time intervals, aliquots taken from the reaction mixture were diluted 10-fold in ice-cold stop-buffer (TBS: 50 mM Tris-HCl, 175 mM NaCl, pH 7.9) containing 20 mM EDTA and 0.5 mg/ml ovalbumin and FXa present in the diluted aliquots was determined with the chromogenic substrate S2765 (Z-D-Arg-Gly-Arg-pNA.2HCl).

### Example 4

### Inhibition of FXa by TFPI

Conversion of the chromogenic substrate S2222 (Bz-Ile-Glu(-OR)-Gly-Arg-pNA.HCl) by FXa was monitored in a Ultra Microplate Reader (Bio-Tek Instruments). A reaction mixture containing CaCl₂, S2222, phospholipid vesicles (20/60/20 DOPS/DOPC/DOPE) with or without protein S was preincubated during 7 min at 37° C. After FXa was added, the increase in absorbance at 405 nm was followed in time. After ˜5 min, TFPI was added to the reaction mixture and the reaction was followed until the rate of chromogenic substrate conversion became constant. Final concentrations in all experiments were 0.2 nM human FXa, 500 μM S2222, and 0 or 10 μM phospholipid vesicles in Hepes-buffered saline containing 5 mg/ml BSA and 3 mM CaCl₂. The dose-dependent effect of TFPI on FXa inhibition was measured in the absence or presence of 100 nM protein S: final concentrations were 0-3.9 nM and 0-7.7 nM TFPI with and without phospholipid vesicles, respectively, and 0-12.7 nM TFPI₁₋₁₆₁.

### Example 5

### Kinetic Analysis

Progress curves of FXa inhibition by TFPI were fitted to the integrated rate equation (d) for slow binding inhibition, generating values for vₒ, vₛ and k_{obs}: vₒ and vₛ are the initial and steady state velocities of pNA formation, respectively, and k_{obs} is the apparent rate constant for the transition from vₒ to vₛ (FXa·TFPI to FXa·TFPI*, equation c). FXa-inhibition at varying TFPI concentrations was measured and Kᵢ values were calculated from a plot of V/vₒ versus the concentration of TFPI, in which V is the rate of pNA formation by FXa in the absence of TFPI (22, 47). The x-intercept of this line is −Kᵢ(1+[S]/Km), in which [S] the concentration chromogenic substrate S2222 in the reaction mixture (0.5 mM). Under the conditions used, the Km value for S2222 conversion by FXa was 1.065 mM. Similarly, Kᵢ* was determined from a plot of V/vₛ versus the concentration of TFPI. Subsequent application of equations (a-b)

k₋₂=k_{obs}·(vₛ/vₒ) (a)

Kᵢ*=Kᵢ·k₋₂/(k₂+k₋₂) (b)

yielded k₂ and k₋₂ (22, 48).

### Example 6

### Effect of Protein S and TFPI on Thrombin Formation

In plasma, in which coagulation was initiated with tissue factor (TF), inhibition of protein S with polyclonal antibodies considerably increased thrombin generation (FIG. 1A). This effect of protein S was independent of APC since all experiments in plasma were performed in the presence of inhibiting antibodies against APC. Calculation of the area under the thrombin-generation curves, which yields the so-called endogenous thrombin potential (ETP), indicated that protein S inhibited thrombin generation approximately two-fold from 735 nM.Ila.min in the absence to 285 nM Ila.min in the presence of protein S. To explore whether the effect of protein S is limited to its free form, plasma was saturated with C4b-binding protein. Addition of a molar excess of purified C4BP to free protein S in plasma resulted in a 40% decrease of the anticoagulant activity of protein S, independent of the concentration of tissue factor used for initiation of coagulation (Table 1).

TABLE 1
Effect of C4BP on the inhibition of thrombin generation by TFPI and
protein S

| TF | Inhibition of ETP by | Inhibition of ETP by | Activity of protein S- |
|---|---|---|---|
| (pM) | protein S (%) | protein S-C4BP (%) | C4BP complex (%) |
| 3.5 | 12.6 | 7.4 | 59 |
| 1.4 | 41.1 | 23.0 | 56 |
| 0.7 | 57.1 | 33.4 | 58 |

ETPs were determined at varying concentrations of TF. The inhibitory effect of protein S-C4BP complex on the ETP was determined by preincubating normal pooled plasma with saturating amounts of purified C4BP (see, Materials and Methods below).

Next, the APC-independent effect of protein S was determined in TFPI-depleted plasma. Thrombin generation in TFPI-depleted plasma was increased compared to normal pooled plasma, which likely reflects increased FXa generation due to the lack of inhibition of the TF/FVIIa complex. In contrast to normal plasma, antibodies against protein S had no effect on thrombin generation in TFPI-depleted plasma. This indicates that protein S does not express APC-independent anticoagulant activity in the absence of TFPI (FIG. 1A) which led to the hypothesis that protein S enhances the ability of TFPI to down-regulate FXa- and thrombin formation during tissue factor-initiated coagulation.

To gain more insight in the interaction between TFPI and protein S, TFPI-depleted plasma was reconstituted with varying amounts of recombinant full length TFPI or with a truncated form of TFPI (TFPI₁₋₁₆₁) that lacks the Kunitz-3 domain and the C-terminus (FIG. 1B). In plasma that contained protein S, thrombin generation decreased with increasing concentrations of full length TFPI (IC₅₀ of ˜1.7 nM) and was fully inhibited at 3.1 nM TFPI, whereas TFPI₁₋₁₆₁, did not show an inhibitory effect. In the absence of protein S, neither full length TFPI nor TFPI₁₋₁₆₁, affected the ETP.

### Example 7

### Inhibition of TF/FVIIa by TFPI and Protein S

TFPI inhibits extrinsic coagulation via a feed-back mechanism that requires the presence of FXa, the product of extrinsic FX activation (12). The first step, in which TFPI binds to and inhibits FXa is rate-limiting (20). The second step, in which FXa/TFPI reacts with TF/FVIIa and forms an inactive quaternary complex, has been reported to proceed at near diffusion-limited rate (20). Since protein S did not inhibit thrombin generation in TFPI-depleted plasma and TFPI lost its anticoagulant activity in the absence of protein S, we hypothesized that protein S stimulates the inhibition of TF/FVIIa-catalyzed factor X activation by TFPI.

This hypothesis was tested in a model system containing purified proteins. FX-activation by TF/FVIIa was followed in time in the absence and presence of TFPI and/or protein S (FIG. 2). In the absence of TFPI, TF/FVIIa-catalyzed FX-activation was linear in time and was not affected by protein S. In the presence of TFPI, the generation of FXa progressively decreased and was fully inhibited after 2 min. When both TFPI and protein S were present, virtually no factor Xa was generated, which indicates that protein S indeed accelerates the inhibition of TF/FVIIa-catalyzed FX-activation by TFPI.

### Example 8

### Inhibition of factor Xa by TFPI and protein S

Theoretically, protein S can accelerate the inhibition of TF/FVIIa-catalyzed FX activation by TFPI by stimulating the formation of the FXa/TFPI and/or the FXa/TFPI/TF/FVIIa (quaternary) complex. Since the formation of the quaternary complex is very fast and diffusion-limited (20) it is unlikely that this step is affected by protein S. Hence, we quantified the effect of protein S on FXa/TFPI complex formation by measuring progress curves of factor Xa inhibition by TFPI. These progress curves were analyzed according to a slow tight-binding mechanism that describes the inhibition of FXa by TFPI (21) (c).

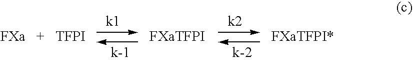

In this mechanism, enzyme (FXa) and inhibitor (TFPI) are in rapid equilibrium and form a complex (FXaTFPI) with a dissociation constant Kᵢ (Kᵢ=k₋₁/k₁=[FXa]·[TFPI]/[FXaTFPI]). The FXa/TFPI complex subsequently slowly isomerizes into a tight complex (FXa/TFPI*) which at final equilibrium results in an overall dissociation constant Kᵢ* that is much lower than Kᵢ (Kᵢ*=[FXa]·[TFPI]/[FXaTFPI+FXaTFPI*]).

Rate constants and dissociation constants for the interaction between TFPI and FXa were determined in reaction mixtures that contained FXa, TFPI and the FXa-specific chromogenic substrate S2222 for monitoring the loss of FXa activity in time. For the mechanism presented in equation (c), the progress curves of S2222 conversion by FXa are described by the integrated rate equation (d) (22):

Aₜ=Aₒ+vₛt+(vₒ−vₛ){1-exp(−k_{obs}t)}/k_{obs} (d)

in which Aₜ and A₀ are the absorbance values at time t and time zero; vₒ and vₛ are the initial velocity and the final steady state velocities of S2222 conversion, respectively, and k_{obs} is the apparent rate constant for the transition from vₒ to vₛ.

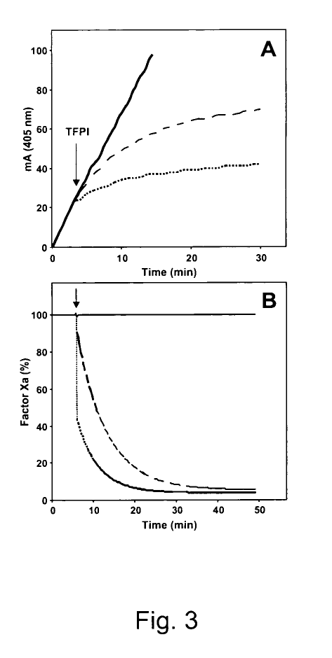

S2222 conversion by FXa was inhibited upon addition of TFPI (1.5 nM) and this inhibition was strongly potentiated by protein S (FIG. 3A). In the absence of TFPI, protein S had no effect on S2222 conversion by FXa indicating that protein S enhances the inhibition of FXa by TFPI. Estimates of free FXa concentrations, obtained from the first derivative of the curves (FIG. 3A), showed that the addition of TFPI resulted in an immediate decrease of FXa activity by ˜10% (FIG. 3B). This indicates that a rapid binding equilibrium was attained in which ˜10% of the FXa present was incorporated in the FXa/TFPI complex. The further decrease of free FXa with time reflects the slow isomerization of FXa/TFPI into the tight FXa/TFPI* complex which causes a continuous re-establishment of equilibrium until finally more than 95% of FXa ended up in a complex with TFPI. In the presence of protein S, the fraction of FXa that was rapidly inhibited by TFPI increased to ˜60% with a similar final equilibrium. This demonstrates that protein S primarily stimulates the formation of the FXa/TFPI complex and has less effect on the isomerization of FXa/TFPI into FXa/TFPI*.

The effect of protein S on FXa inhibition by TFPI was further explored by measuring progress curves of FXa inhibition at varying TFPI concentrations (0-3.9 nM) both in the absence and presence of 100 nM protein S. Fitting the experimental data to equation (d) yielded values for vₒ, vₛ and k_{obs} at each TFPI concentration from which the rate constants k₊₂ and k₋₂ and the dissociation constants Kᵢ and Kᵢ* were calculated (see Materials and Methods).

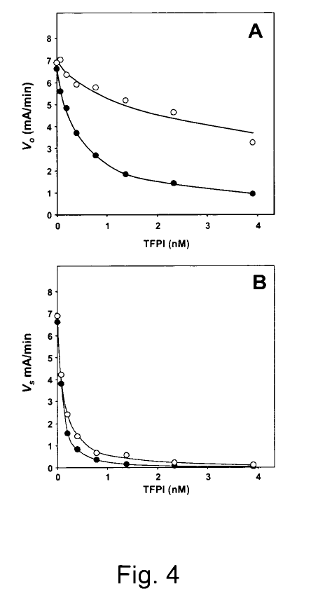

Both in the presence and absence of protein S, the initial velocity vₒ decreased with increasing concentrations TFPI (FIG. 4A). However, the decrease of the vₒ was more pronounced in the presence of protein S, supporting the concept that protein S promotes the formation of the FXa/TFPI complex. The dissociation constant Kᵢ of this complex decreased from 4.4 nM in the absence of protein S to 0.5 nM in the presence of protein S (Table 2). The final equilibrium rate (vₛ) also decreased with increasing concentrations TFPI, but protein S had much less effect on vₛ than on vₒ, (FIG. 4B). From the variation of vₛ as function of the TFPI concentration, Kᵢ* values were calculated which were 0.05 nM in the absence and 0.02 nM in the presence of protein S, respectively (Table 2). Comparison of k₊₂ determined in the absence (k₊₂=2.5 min⁻¹) and presence of protein S (k₊₂=0.72 min⁻¹) indicated that protein S actually slowed down the transition of FXa/TFPI into FXa/TFPI*. The reverse reaction described by k₋₂ was not influenced by protein S (Table 2).

TABLE 2
Kinetic constants for the inhibition of
FXa by TFPI with or without protein S.

| Addition | TFPI type | Kᵢ (nM) | Ki* (nM) | k₋₂, min⁻¹ | k₊₂, min⁻¹ |
|---|---|---|---|---|---|
| None | TFPI_{fl} | 8.3 | 0.08 | 0.018 | 1.84 |
| Protein S | TFPI_{fl} | 10.9 | 0.12 | 0.009 | 0.75 |
| PL | TFPI_{fl} | 4.4 | 0.05 | 0.030 | 2.49 |
| PL + Protein S | TFPI_{fl} | 0.5 | 0.02 | 0.028 | 0.72 |
| PL | TFPI₁₋₁₆₁ | 39.0 | 0.21 | 0.010 | 11.88 |
| PL + Protein S | TFPI₁₋₁₆₁ | 42.3 | 0.10 | 0.013 | 5.52 |

PL = phospholipid vesicles 20/60/20 DOPS/DOPC/DOPE;
TFPI_{fl} = full length TFPI;
TFPI₁₋₁₆₁ is a truncated form of TFPI that lacks the third Kunitz domain and the C-terminus.

The stimulatory effect of protein S on FXa inhibition by TFPI required the presence of anionic phospholipids (Table 2). In the absence of phospholipid, protein S hardly influenced the Kᵢ (8.3 nM without and 10.9 nM with protein S, Table 2). The finding that TFPI was a relatively poor inhibitor of FXa in reaction mixtures containing calcium ions but no phospholipids is in agreement with literature (21). The Kᵢ's determined for FXa inhibition by TFPI₁₋₁₆₁ explain the observed lack of inhibitory activity of TFPI₁₋₁₆₁ on thrombin formation in plasma (FIG. 1). The fact that protein S had no effect on complex formation between FXa and TFPI₁₋₁₆₁ (Table 2) indicates that the Kunitz 3 and/or the C-terminal domain of TFPI are involved in protein S-dependent stimulation of TFPI-activity. The effect of protein S on initial FXa/TFPI complex formation in a model system using fixed amounts of purified factor Xa was half-maximal at 45 nM protein S (data not shown) and reached optimal levels around the free protein S concentration present in plasma (150 nM). However, in a more physiologic plasma model system in which factor Xa was generated by TF-FVIIa (19), a dose-dependent decrease of the ETP was observed with increasing concentrations of protein S over the whole possible range (0-100%) of protein S concentrations in plasma. In this respect, any change of protein S concentration in plasma will be able to affect the regulation of thrombin generation.

### REFERENCES

1. Levi, M. & Ten Cate, H. (1999) N Engl J Med 341, 586-92.
2. Tremoli, E., Camera, M., Toschi, V. & Colli, S. (1999) Atherosclerosis 144, 273-83.
3. Riewald, M. & Ruf, W. (2001) Proc Natl Acad Sci USA 98, 7742-7.
4. Camerer, E., Huang, W. & Coughlin, S. R. (2000) Proc Natl Acad Sci USA 97, 5255-60.
5. Pawlinski, R., Pedersen, B., Schabbauer, G., Tencati, M., Holscher, T., Boisvert, W., Andrade-Gordon, P., Frank, R. D. & Mackman, N. (2004) Blood 103, 1342-7.
6. Belting, M., Dorrell, M. I., Sandgren, S., Aguilar, E., Ahamed, J., Dorfleutner, A., Carmeliet, P., Mueller, B. M., Friedlander, M. & Ruf, W. (2004) Nat Med 10, 502-9.
7. Carmeliet, P., Mackman, N., Moons, L., Luther, T., Gressens, P., Van Vlaenderen, I., Demunck, H., Kasper, M., Breier, G., Evrard, P., Muller, M., Risau, W., Edgington, T. & Collen, D. (1996) Nature 383, 73-5.
8. Rosendaal, F. R. (1999) Thromb Haemost 82, 610-9.
9. Makris, M., Leach, M., Beauchamp, N. J., Daly, M. E., Cooper, P. C., Hampton, K. K., Bayliss, P., Peake, I. R., Miller, G. J. & Preston, F. E. (2000) Blood 95, 1935-41.
10. Dahm. A., Van Hylckama Vlieg, A., Bendz, B., Rosendaal, F., Bertina, R. M. & Sandset, P. M. (2003) Blood 101, 4387-92.
11. Broze, G. J., Jr. (1995) Annu Rev Med 46, 103-12.
12. Girard, T. J., Warren, L. A., Novotny, W. F., Likert, K. M., Brown, S. G., Miletich, J. P. & Broze, G. J., Jr. (1989) Nature 338, 518-20.
13. Esmon, C. T. (1989) J Biol Chem 264, 4743-6.
14. Rosing, J., Hoekema, L., Nicolaes, G. A., Thomassen, M. C., Hemker, H. C., Varadi, K., Schwarz, H. P. & Tans, G. (1995) J Biol Chem 270, 27852-8.
15. O'Brien, L. M., Mastri, M. & Fay, P. J. (2000) Blood 95, 1714-20.
16. Heeb, M. J., Mesters, R. M., Tans, G., Rosing, J. & Griffin, J. H. (1993) J Biol Chem 268, 2872-7.
17. Heeb, M. J., Rosing, J., Bakker, H. M., Fernandez, J. A., Tans, G. & Griffin, J. H. (1994) Proc Nat Acad Sci USA 91, 2728-32.
18. Hackeng, T. M., van 't Veer, C., Meijers, J. C. & Bouma, B. N. (1994) J Biol Chem 269, 21051-8.
19. Sere, K. M., Rosing, J. & Hackeng, T. M. (2004) Blood 104, 3624-30.
20. Baugh, R. J., Broze, G. J., Jr. & Krishnaswamy, S. (1998) J Biol Chem 273, 4378-86.
21. Huang, Z. F., Wun, T. C. & Broze, G. J., Jr. (1993) J Biol Chem 268, 26950-5.
22. Morrison, J. F. & Walsh, C. T. (1988) Adv Enzymol Relat Areas Mol Biol 61, 201-301.
23. Mitchell, C. A., Kelemen, S. M. & Salem, H. H. (1988) Thromb Haemost 60, 298-304.
24. Sere, K. M., Janssen, M. P., Willems, G. M., Tans, G., Rosing, J. & Hackeng, T. M. (2001) Biochemistry 40, 8852-60.
25. Piro, O. & Broze, G. J., Jr. (2004) Circulation 110, 3567-72.
26. Valentin, S. & Schousboe, I. (1996) Thromb Haemost 75, 796-800.
27. Lockett, J. M. & Mast, A. E. (2002) Biochemistry 41, 4989-97.
28. Lindhout, T., Willems, G., Blezer, R. & Hemker, H. C. (1994) Biochem J 297 (Pt 1), 131-6.
29. Wesselschmidt, R., Likert, K., Girard, T., Wun, T. C. & Broze, G. J., Jr. (1992) Blood 79, 2004-10.
30. Huang, Z. F., Higuchi, D., Lasky, N. & Broze, G. J., Jr. (1997) Blood 90, 944-51.
31. Mahasandana, C., Suvatte, V., Marlar, R. A., Manco-Johnson, M. J., Jacobson, L. J. & Hathaway, W. E. (1990) Lancet 335, 61-2.
32. Hansen, J. B., Huseby, K. R., Huseby, N. E., Ezban, M. & Nordoy, A. (1997) Thromb Res 85, 413-25.
33. Hockin, M. F., Jones, K. C., Everse, S. J. & Mann, K. G. (2002) J Biol Chem 277, 18322-33.
34. Koyama, T., Nishida, K., Ohdama, S., Sawada, M., Murakami, N., Hirosawa, S., Kuriyama, R., Matsuzawa, K., Hasegawa, R. & Aoki, N. (1994) Br J Haematol 87, 343-7.
35. Mueller, B. M., Reisfeld, R. A., Edgington, T. S. & Ruf, W. (1992) Proc Natl Acad Sci USA 89, 11832-6.
36. Riewald, M., Kravchenko, V. V., Petrovan, R. J., O'Brien, P. J., Brass, L. F., Ulevitch, R. J. & Ruf, W. (2001) Blood 97, 3109-16.
37. Hjortoe, G. M., Petersen, L. C., Albrektsen, T., Sorensen, B. B., Norby, P. L., Mandal, S. K., Pendurthi, U. R. & Rao, L. V. (2004) Blood 103, 3029-37.
38. Ahamed, J. & Ruf, W. (2004) J Biol Chem 279, 23038-44.
39. Ahamed, J., Belting, M. & Ruf, W. (2005) Blood 105, 2384-91.
40. Salemink, I., Franssen, J., Willems, G. M., Hemker, H. C. & Lindhout, T. (1999) J Biol Chem 274, 28225-32.
41. Diaz-Collier, J. A., Palmier, M. O., Kretzmer, K. K., Bishop, B. F., Combs, R. G., Obukowicz, M. G., Frazier, R. B., Bild, G. S., Joy, W. D., Hill, S. R. & et al. (1994) Thromb Haemost 71, 339-46.
42. Petersen, J. G., Meyn, G., Rasmussen, J. S., Petersen, J., Bjorn, S. E., Jonassen, I., Christiansen, L. & Nordfang, O. (1993) J Biol Chem 268, 13344-51.
43. Girard, T. J. & Broze, G. J., Jr. (1993) Methods Enzymol 222, 195-209.
44. van 't Veer, C., Hackeng, T. M., Delahaye, C., Sixma, J. J. & Bouma, B. N. (1994) Blood 84, 1132-42.
45. Sandset, P. M., Larsen, M. L., Abildgaard, U., Lindahl, A. K. & Odegaard, O. R. (1991) Blood Coagul Fibrinolysis 2, 425-33.
46. Hemker, H. C., Giesen, P., Al Dieri, R., Regnault, V., de Smedt, E., Wagenvoord, R., Lecompte, T. & Beguin, S. (2003) Pathophysiol Haemost Thromb 33, 4-15.
47. Longstaff, C. & Gaffney, P. J. (1991) Biochemistry 30, 979-86.
48. Morrison, J. F. (1982) Trends Biochem Sci 7, 102-5.

1. A method for the identification of a compound that improves or decreases the inhibitory effect of TFPI on tissue factor activity and/or Factor Xa activity and/or thrombin formation.

2. The method according to claim 1 wherein the inhibitory effect of TFPI is measured in an assay for tissue factor activity and/or Factor Xa activity, and/or an assay for thrombin formation, in the presence of protein S.

3. The method according to claim 1 wherein the inhibitory effect of TFPI is measured in the presence and absence of a potential TFPI agonist or antagonist and the level of TFPI inhibition in the presence of the potential agonist or antagonist is compared with the level of TFPI inhibition in the absence of the potential TFPI agonist or antagonist.

4. The method according to claim 1 wherein the potential TFPI agonist or antagonist is selected from the group consisting of antibodies against Protein S, antibodies against TFPI, fragments or analogues of TFPI, fragments or analogues of Protein S, fragments or analogues of Factor Xa and fragments or analogues of Tissue Factor.

5. The method according to claim 4 wherein the antibodies are monoclonal, polyclonal, bi-specific or single chain.

6. The method according to claim 5 wherein the bi-specific antibody has affinity for Protein S as well as TFPI.

7. The method according to claim 1 wherein tissue factor activity and/or Factor Xa activity is measured using a chromogenic or fluorogenic substrate.

8. The method according to claim 1 for the identification of a compound that is capable of increasing or decreasing the co-factor activity of Protein S in TFPi-mediated inhibition of tissue factor activity and/or Factor Xa activity and/or thrombin formation.

9. The method according to claim 1 wherein the compound that specifically improves or decreases the inhibitory effect of TFPI on tissue factor activity and/or Factor Xa activity and/or thrombin formation does not interfere with the co-factor activity of Protein S on the APC system.

10. A method for the regulation of tissue factor activity and/or Factor Xa activity by influencing the interaction between Protein S and TFPI and/or Factor Xa.

11. The method for the regulation of tissue factor activity and/or Factor Xa activity by influencing the interaction between Protein S and TFPI and/or Factor Xa wherein a compound such as an agonist or antagonist of TFPI is used that is identifiable or identified by a method according to claim 1.

12. The use of a TFPI and/or Protein S antagonist identifiable or identified by a method according to claim 1 for the preparation of a medicament for increasing the coagulation potential of blood.

13. The use according to claim 12 for the treatment of patients with a low level of tissue factor.

14. The use according to claim 12 for the treatment of patients with bleeding disorders such as hemophilia.

15. The use of a TFPI and/or Protein S agonist identifiable or identified by a method to claim 1 for the preparation of a medicament for decreasing the coagulation potential of blood.

16. The use according to claim 15 for the treatment of patients with thrombotic disorders such as deep venous thrombosis.

17. The method for the preparation of a medicament for the treatment of thrombotic disorders comprising the steps of:
a. Identifying a compound that improves the inhibitory effect of TFPI on tissue factor activity and/or Factor Xa activity and/or thrombin formation by a method according to claim 1; and
b. mixing the compound identified in step a) with a pharmaceutically acceptable carrier.

18. The method for the preparation of a medicament for the treatment of bleeding disorders comprising the steps of:
a. Identifying a compound that decreases the inhibitory effect of TFPI on tissue factor activity and/or Factor Xa activity and/or thrombin formation by a method according to claims 1; and
b. mixing the compound identified in step a) with a pharmaceutically acceptable carrier.

19. A use of a compound capable of increasing or decreasing the co-factor activity of Protein S in TFPI-mediated inhibition of tissue factor for the preparation of a medicament for the treatment of a disease associated with tissue factor activity in blood.

20. The use according to claim 19 wherein the disease associated with tissue factor activity is selected from the group consisting of disorders wherein the coagulation is impaired so that the tendency to clot is too strong or too weak.

21. The use according to claim 19 wherein the disease associated with tissue factor activity is selected from the group consisting of thrombosis, haemostasis, cancer, inflammation, cardiovascular disorders, hemophilia and related disorders.
